(19) United States
(12) Patent Application Publication
(10) Pub. No.: US 2004/0073162 A1
(43) Pub. Date: Apr. 15, 2004

(54) Balloon construction for occlusion device

(76) Inventors: Jefferey C. Bleam, Boulder Creek, CA (US); Juan T. Domingo, Union City, CA (US); Ketan P. Muni, San Jose, CA (US); Sivette Lam, Santa Rosa, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/446,634

(22) Filed: May 28, 2003

Related U.S. Application Data
(60) Provisional application No. 60/384,149, filed on May 29, 2002.

(51) Int. Cl. A61M 29/00

(52) U.S. Cl. 604/103

(57) ABSTRACT

A balloon catheter construction is disclosed wherein bands are used to mechanically attach a balloon to a catheter shaft. The bands provide a mechanical seal that secures the balloon to the catheter shaft without the use of an adhesive and prevents fluid loss from the interior of the balloon. Fillets may be provided at the ends of the balloon and band to provide a smooth transition region between the shaft and the balloon for facilitating advancement through a blood vessel.

### RELATED APPLICATIONS

The present application claims priority under 35 U.S.C. § 19(e) to U.S. Provisional Application No. 60/384,149, filed May 29, 2002, which is hereby incorporated by reference in its entirety.

### BACKGROUND OF THE INVENTION

1. Field of Invention

The present invention relates generally to medical catheters for use in intravascular procedures and, in particular, to a balloon construction for an occlusion device.

2. Description of Related Art

Medical catheters such as balloon catheters have proven efficacious in treating a wide variety of blood vessel disorders. Moreover, these types of catheters have permitted technicians to treat disorders with minimally invasive procedures that, in the past, would have required complex and perhaps life-threatening surgeries. In one example, medical catheters are provided with an inflatable balloon along the distal end portion for use as a temporary occlusion device. In this application, the “balloon catheter” is advanced through a patient's vasculature and the balloon is inflated in a blood vessel distal to a treatment site. The inflated balloon prevents emboli and other material from migrating downstream during treatment on the blood vessel. In another application, a similar type of balloon catheter is used in a common medical procedure, called angioplasty, wherein the device is advanced through a patient's vasculature to a stenotic lesion (i.e., a clogged artery) and the balloon is inflated to reopen the vessel.

The manufacture of balloon catheters, such as the types described above, typically involves placing a tubular member over the distal end portion of a catheter shaft (e.g., a guidewire) and attaching the proximal and distal ends of the tubular member to the catheter shaft with an adhesive material. After curing, the adhesive material provides a fluid tight seal at the proximal and distal ends of the tubular member. As a result, the interior region of the tubular member can be filled with a pressurized fluid, thereby providing an inflatable “balloon” on the catheter shaft.

Although balloon catheters constructed in this manner have met with considerable success, there are a variety of drawbacks associated with the use of an adhesive material for attaching the ends of the tubular member to the catheter shaft. For example, during construction, the tubular member must be formed with an inner diameter that is substantially larger than the outer diameter of the catheter shaft. The large inner diameter in the tubular member is required such that the adhesive material may be adequately applied in the annular gap between the catheter shaft and tubular member. In practice, this requirement is disadvantageous during the construction of low profile balloons.

Another significant drawback is the inability to adequately control the distribution of adhesive material during manufacture of the balloon catheter. The movement or flow of adhesive in the gap between the catheter shaft and the tubular member, often referred to as “wicking,” is often unpredictable. The unpredictable nature of wicking is disadvantageous because either too much or too little wicking can adversely affect the performance of the balloon catheter. For example, balloon catheters manufactured in the same manner can have different qualities, thereby making it difficult for the clinician to precisely inflate the balloon to a desired dimension during treatment of a blood vessel.

Another serious drawback is the possibility of a partial or complete failure of the adhesive bond between the tubular member and the catheter shaft. When the interior region of the tubular member is filled with pressurized fluid, large stresses are applied along the edges of the adhesive. Likewise, bending of the catheter shaft produces relative movement between the tubular member and the catheter that produces large stresses along the adhesive. During use, these stresses can lead to a complete failure of the adhesive bond. In practice, it has been found that a failure is particularly likely to occur if the balloon catheter has undergone multiple inflation cycles.

Yet another drawback is the rigidity of the adhesive material after curing. The rigidity of the adhesive material is undesirable because it can substantially limit the flexibility of the catheter shaft. The limited flexibility is particularly undesirable because it can impede the physician's ability to advance the catheter through a patient's vasculature to a treatment site.

Accordingly, what is needed is an improved balloon catheter construction wherein a tubular member is attached along a catheter shaft in a manner that overcomes some or all of the above drawbacks. It is desirable that such a balloon catheter construction be capable of withstanding significant bending and numerous inflation cycles while maintaining a fluid tight seal between the tubular member and catheter shaft. It is also desirable that such a balloon catheter construction provides a balloon with a highly predictable overall length and diameter when inflated. It is also desirable that such a balloon catheter construction be adaptable for providing a variety of different balloon shapes and sizes. It is also desirable that such a balloon catheter construction be well-suited for the manufacture of a low profile catheter for advancement into small regions of a patient's vasculature. It is also desirable that such a balloon catheter construction be capable of providing an occlusion device for use in a blood vessel.

### SUMMARY OF THE INVENTION

Various embodiments of the present invention provide a balloon catheter having bands for mechanically bonding the ends of a tubular member (i.e., balloon) to the catheter shaft in a secure and predictable manner. A significant advantage of these embodiments is the ability to provide a balloon catheter having a very small profile. Another significant advantage is the improved consistency of construction that is achieved by eliminating the unpredictable wicking of an adhesive material. Yet another feature is the ability to withstand large stresses. Yet another feature is the improved flexibility of the catheter shaft.

In one embodiment, two or more bands may be provided along a tubular member. Balloon catheters constructed in this manner may be provided with a variety of different shapes and sizes.

In another embodiment, fillet tapers are provided along the ends of each band to form a smooth transition region between the band and the shaft. The fillet tapers reduce trauma to the surrounding tissue during advancement of the balloon catheter to a treatment site.

In another embodiment, one or more bands may be used in combination with an adhesive to further enhance the fluid tight seal. When bands are used with an adhesive, the catheter shaft is desirably constructed with a selectively reduced compliance for reducing stresses on the adhesive and increasing the peel strength to further improve the structural integrity of the balloon catheter.

In another embodiment, the bands may be provided with a radiopaque material for enhancing visibility of the balloon catheter during advancement through a patient's vasculature.

In another embodiment, an occlusion balloon guidewire is provided for occluding a blood vessel distal to a treatment site. The occlusion balloon guidewire generally comprises an elongate body (e.g., guidewire), a compliant balloon disposed along the distal end portion of the elongate body and first and second bands disposed over the proximal and distal ends of the compliant balloon. The bands may be used to secure the balloon to the elongate body without the use of an adhesive material while providing a fluid tight seal in the interior region of the balloon.

In another embodiment, various methods of constructing a balloon catheter are provided wherein bands are used to mechanically bond the expandable tubular member (i.e., balloon) to the catheter shaft. One method desirably includes placing a tubular member over a catheter shaft, placing first and second lengths of shrink tubing over the ends of the tubular member and shrinking the shrink tubing by application of heat. As the shrink tubing reduces in diameter, the ends of the tubular member become tightly compressed between the bands and the catheter shaft for securing the tubular member to the catheter shaft and thereby providing an inflatable balloon.

### BRIEF DESCRIPTION OF THE DRAWINGS

FIG. 1 is a side view of an occlusion balloon guidewire that can be used in accordance with embodiments of the present invention.

FIG. 2A is a partial cross-sectional view of a valve mechanism incorporated into the guidewire of FIG. 1.

FIG. 2B is an enlarged view of the valve mechanism of FIG. 2A, showing the valve mechanism in an open position (and a closed position shown in

FIG. 3 is a side cross-sectional view of the distal end portion of a balloon catheter schematically illustrating the use of bands for attaching a balloon to a catheter shaft in accordance with one embodiment of the present invention.

FIG. 3A is a cross-sectional view taken along line 3A-3A of FIG. 3 illustrating the proximal band surrounding the proximal end of the balloon.

FIG. 3B is a cross-sectional view taken along line 3B-3B of FIG. 3 illustrating the balloon position relative to the catheter shaft while in a deflated condition.

FIG. 4 is a side cross-sectional view of the balloon catheter of FIG. 3 with the balloon inflated and further comprising a core wire with a flexible tip.

FIG. 4A is a cross-sectional view taken along line 4A-4A of FIG. 4 illustrating the balloon in an inflated condition.

FIG. 4B is a side view of a balloon catheter illustrating the compliant and non-compliant regions of the balloon.

FIG. 5 is a side cross-sectional view illustrating the distal end portion of one embodiment of a fully assembled balloon catheter.

FIG. 5A is a side cross-sectional view illustrating the balloon catheter of FIG. 5 in an inflated condition.

FIG. 6 is a side cross-sectional view of an alternative embodiment of a balloon catheter further comprising a third band for creating a double balloon configuration.

FIG. 7 is a side cross-sectional view of another alternative embodiment wherein the catheter shaft is formed with a reduced diameter in the regions adjacent the bands for reducing the profile of the balloon catheter.

FIG. 8 is a side cross-sectional view of another alternative embodiment wherein bands are formed as part of the catheter shaft.

FIGS. 9A and 9B are side views of another embodiment of a band formed with a locking mechanism.

### DETAILED DESCRIPTION OF THE PREFERRED EMBODIMENTS

Various embodiments of the present invention depict balloon catheters having improved bond integrity and uniformity in inflation. Although the disclosed embodiments are depicted and discussed in the context of being part of a simple occlusive device having a single lumen, it should be appreciated that the principles and aspects of these embodiments are applicable to other devices having structures and functionalities not discussed herein. Thus, the embodiments are not only applicable to catheters having occlusion balloons made of compliant materials such as C-Flex, latex or silicone, but are also applicable to to catheters used for dilatation balloons made of non-compliant materials such as polyethylene terephthalate, and other balloon catheters. The manner of adapting the embodiments described herein to these various structures and functionalities will become apparent to those of skill in the art in view of the description that follows.

### I. Overview of an Occlusion Balloon Guidewire

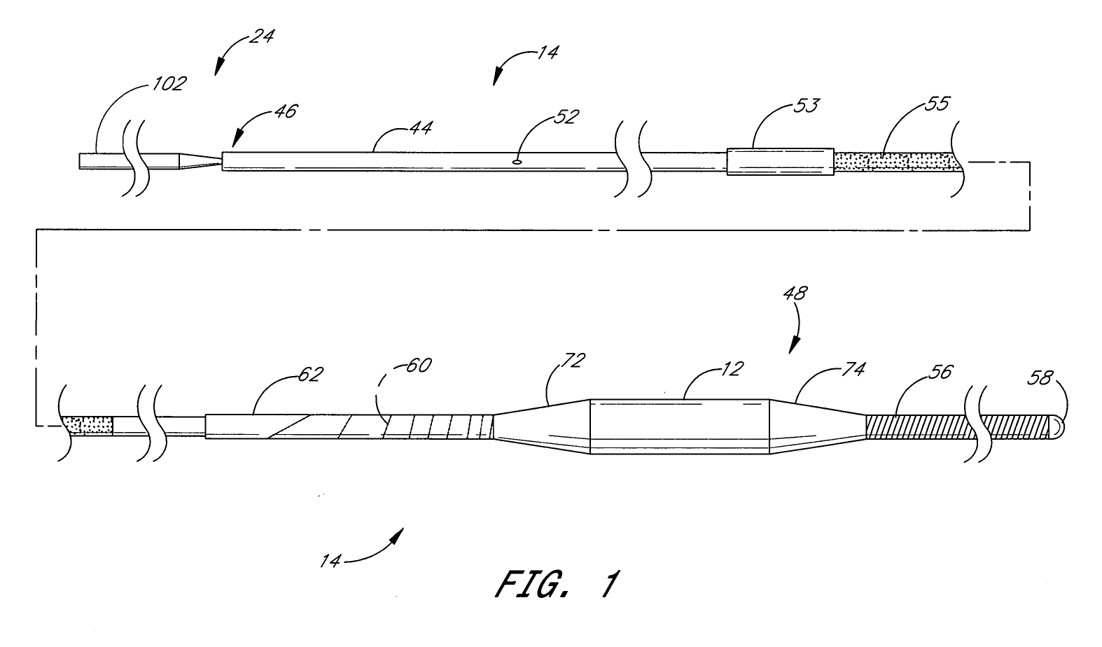

FIG. 1 illustrates one embodiment of an occlusive device that can be used in combination with the balloon catheter construction described below. In the embodiment shown, the occlusive device is an occlusion balloon guidewire. The illustrated occlusion balloon guidewire 14 performs the function of occluding a vessel and allowing for the slidable insertion or advancement of various other catheters and devices. The term “guidewire” or “occlusion balloon guidewire” as used herein is intended to include both guidewires and a wide variety of other catheters with these desired characteristics. One suitable guidewire system is available from Medtronic AVE under the name GUARDWIRE PLUS™.

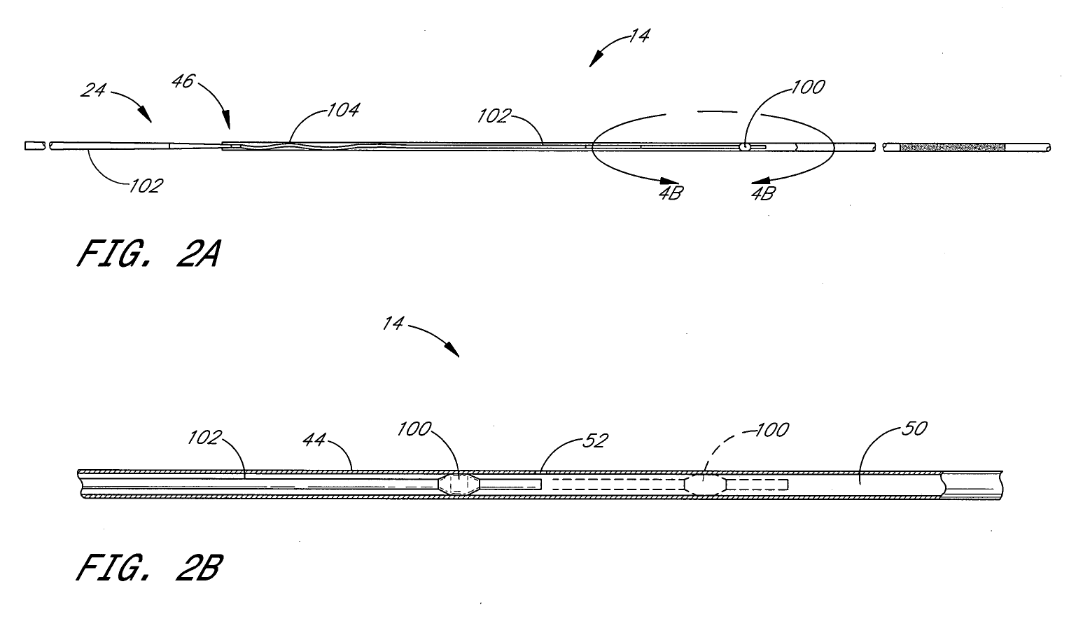

As shown in FIG. 1, an occlusion balloon guidewire 14 generally comprises an elongate flexible tubular body 44 extending between a proximal control end 46, corresponding to a proximal section of the tubular body 44, and a distal functional end 48, corresponding to a distal section of tubular body 44. Tubular body 44 has a central lumen 50, shown in FIG. 2B, which extends between the proximal and distal ends. An inflation port 52, shown also in FIGS. 2A and 2B described below, is provided on tubular body 44 near the proximal end 46. Inflation port 52 is in fluid communication with lumen 50 such that fluid passing through inflation port 52 into or out of the lumen 50 may be used to inflate or deflate an inflatable balloon 12 in communication with lumen 50.

A wire 102, as described below, is inserted into the proximal end 46 of the tubular body 44 to control inflation of a balloon 12 mounted on the distal end of the tubular body through inflation port 52. A marker 53, which may be made of gold, is placed over the tubular body 44 distal to the inflation port 52. Distal to the marker 53, a nonuniform coating 55 of polymer material, for example polytetrafluoroethylene (PTFE), is applied to the tubular body 44, terminating proximal to a shrink tubing 62. The shrink tubing 62 extends up to and within the balloon 12, and covers spiral cuts 60 formed in the tubular body 44. These spiral cuts 60 extend to a location between the proximal and distal ends of the balloon 12, and distal to the shrink tubing 62, such that fluid delivered through the lumen 50 enters the balloon 12 through the turns of the cuts 60. Adhesive tapers 72 and 74 extend from the proximal and distal ends of the balloon 12, respectively. The proximal taper 72 extends from the proximal end of the balloon to the shrink tubing 62 on the tubular body 44, while the distal taper 74 extends to coils 56 extending from the distal end 48 of the tubular body 44. The coils 56 terminate in a rounded cap 58.

Other details regarding construction of the balloon guidewire described above as well as similar devices may be found in assignee's U.S. Pat. No. 6,068,623, U.S. Pat. No. 6,228,072, U.S. Pat. No. 6,500,147 and assignee's application entitled FLEXIBLE CATHETER WITH BALLOON SEAL BANDS, application Ser. No. 09/653,217, filed Aug. 31, 2000, now abandoned, each of which is hereby incorporated by reference in its entirety.

As shown in FIGS. 2A and 2B, the wire 102 is inserted into the lumen 50 of the hollow tubular body 44 and has a proximal end that is positioned outside of the hollow tubular body proximal to the proximal end 46. A movable sealer portion 100 is attached at a distal end of the wire 102 and is positioned within the inflation lumen 50 of the guidewire 14. In one embodiment, the wire 102 includes a zig-zag portion 104, which may be formed integrally or separate from the wire 102, the zig-zag portion 104 being proximal to the sealer portion 100 and providing a retention force to the wire 102 due to frictional engagement with the walls of the lumen 50. The sealer portion 100 forms a fluid tight seal with the inflation lumen 50 by firmly contacting the entire circumference of a section of the inflation lumen 50.

As shown in FIGS. 2A and 2B, the combination of the wire 102, the tubular body 44 having lumen 50, the inflation port 52 and the sealer portion 100 together form one embodiment of a valve mechanism 24. The sealer portion 100 may be positioned proximally of the inflation port 52 on the guidewire as shown in FIG. 2B, to establish an unrestricted fluid pathway between the inflation port 52 and the inflatable balloon 12 on the distal end. In this configuration, the valve mechanism 24 is “open.” As desired, the clinician may move the sealer portion 100 to a position at or distal of the inflation port 52, as shown in phantom in FIG. 2B, thereby preventing any fluid from being introduced into or withdrawn from the lumen 50 via the inflation port 52. In this configuration, the valve mechanism 24 is “closed.” The valve mechanism 24 in the embodiment shown is considered “low profile” because the wire 102 is no larger in cross-sectional diameter than the guidewire 14 itself. Further details of these features and other assemblies, as well as adapters that can be used to manipulate the valve mechanism 24, may be found in assignee's U.S. Pat. No. 6,050,972, and assignee's copending application entitled IMPROVED CATHETER INFLATION DEVICE AND ADAPTER, Ser. No. 10/348,046, filed Jan. 17, 2003, the entirety of both which are hereby incorporated by reference.

The occlusive device described above advantageously enables an exchange of catheters over the guidewire while the balloon is inflated, for example, to isolate particles within a blood vessel. For example, a therapy catheter such as a PTCA or stent delivery catheter can be delivered over the guidewire to perform treatment, and then be exchanged with an aspiration catheter to remove particles from the vessel. Further details of this exchange and various treatment procedures are described in U.S. Pat. No. 6,544,276 and U.S. Pat. No. 6,135,991, the entirety of both of which are hereby incorporated by reference.

### II. Balloon Construction

The manufacture of a balloon catheter, such as the type described above, has typically involved placing a tubular member (i.e., balloon) over the distal end portion of a catheter shaft and bonding the ends of the tubular member to the catheter shaft with an adhesive material. However, in practice, it has been found that the use of an adhesive material for attaching the tubular member to the catheter shaft has a variety of significant drawbacks. Therefore, a need exists for an improved balloon catheter construction and method of manufacture.

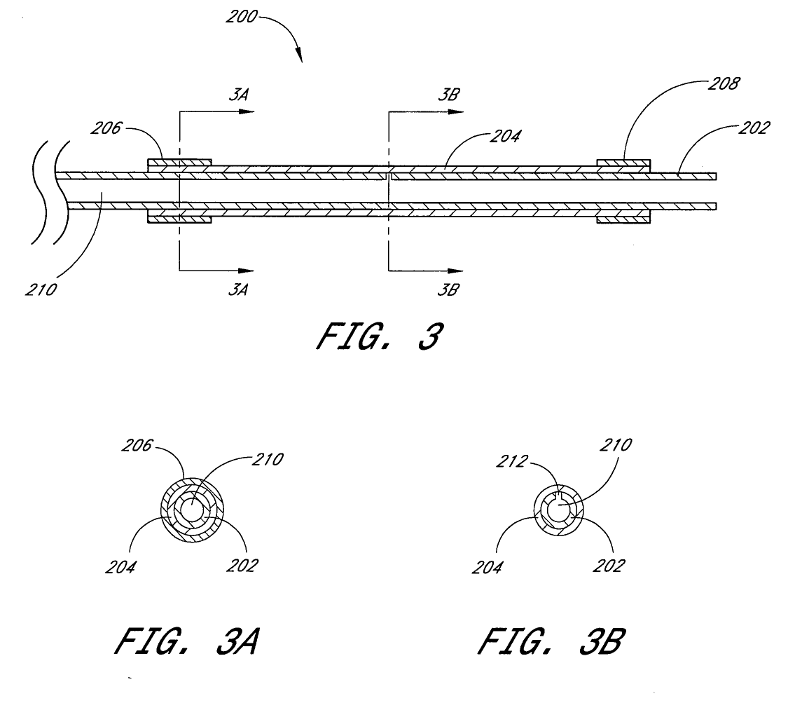

Various embodiments of a balloon catheter will now be described with reference to FIGS. 3 through 9B. The improved balloon catheter construction provides improved bond integrity between the balloon and catheter shaft and overcomes most or all of the drawbacks associated with the use of an adhesive material. Various embodiments may be used as part of the occlusion balloon guidewire system described above with reference to FIGS. 1 through 2B. However, it should be appreciated that the principles and aspects of these embodiments may be used in the construction of a wide variety of other balloon catheters, such as, for example, angioplasty balloon catheters.

Referring now to FIG. 3, one embodiment of a balloon catheter 200 (shown as a subassembly) generally comprises an elongate catheter shaft 202, a tubular member 204, a proximal band 206 and a distal band 208. The bands 206, 208 are generally tubular in shape and are adapted to encircle the outer diameter of the tubular member 204 along the proximal and distal end portions. When applied, the bands apply a sufficient compressive force to securely attach the tubular member to the catheter shaft and produce a fluid tight seal between the catheter shaft and the tubular member. Various embodiments of the bands will be described in more detail below.

The catheter shaft 202 used with the balloon catheter 200 may comprise a hollow hypotube as is known in the art, and as described in U.S. Pat. No. 6,068,623, incorporated by reference herein. The outer diameter of the catheter shaft is preferably sized for insertion into the vasculature of a patient through an insertion site in the skin. One embodiment of the catheter shaft may be particularly well suited for advancement into treatment sites within small blood vessels, such as those located in the brain. When provided as a guidewire, the catheter shaft may be made of nitinol or any other suitable material.

FIGS. 3A and 3B are cross-sectional views of the distal end portion of the balloon catheter 200 with the tubular member in a deflated condition. FIG. 3A illustrates the cross-section along the region of the proximal band 206. FIG. 3B illustrates the cross-section along the region between the bands 206, 208. An inflation lumen 210 extends along the longitudinal axis of the catheter shaft 202. An exit port 212 is provided along a side wall of the catheter shaft 202.

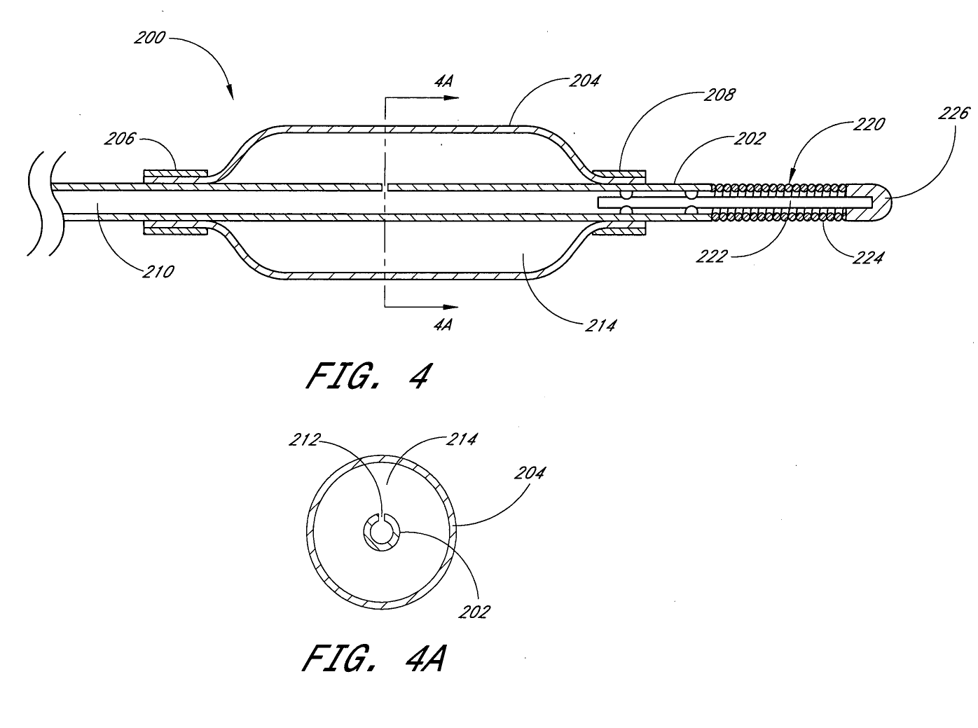

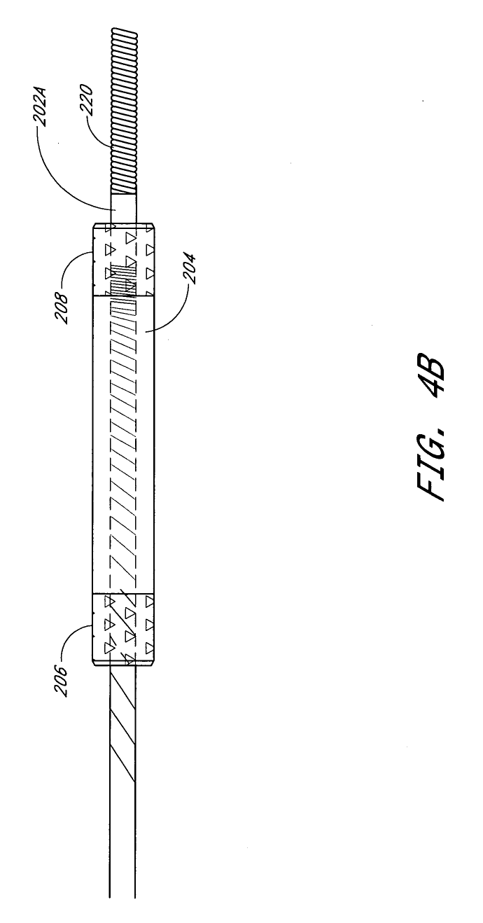

FIG. 4 is a side cross-sectional view of the balloon catheter 200 of FIG. 3, now fully assembled, with the tubular member 204 in an inflated condition. FIG. 4A is a cross-sectional view illustrating an interior region 214 of the tubular member that is filled with a pressurized fluid. The interior region 214 is in fluid communication with the inflation lumen 210. Fluid enters the interior region 214 of the tubular member 204 through the exit port 212 provided along a side wall of the catheter shaft 202. The exit port 212 may be located anywhere between the proximal and distal ends of the tubular member 204. The exit port 212 may take a variety of different forms, such as, for example, one or more holes or a spiral slit formed along the catheter shaft. Any exit port configured for allowing fluid to exit the inflation lumen and enter the interior region of the “balloon” is contemplated.

The embodiment illustrated in FIG. 4 further includes a flexible guide tip 220. The flexible guide tip comprises a core wire 222 that extends distally from the distal end of the catheter shaft 202 and a distal coil 224 that is disposed around the core wire. The flexible guide tip 220 is adapted to facilitate the advancement of the balloon catheter 200 through the vasculature of the patient. A rounded cap 226 may be provided over the distal end of the guide tip such that the device will not damage the patient's tissue during advancement through the patient's vasculature.

The tubular member 204 is preferably located over the distal end portion of the catheter shaft 202 in a substantially concentric arrangement. The tubular member 204 may be constructed from any suitable material as is known in the art. In one preferred embodiment, at least a portion of the balloon is made of a compliant or elastomeric material, such as latex or C-Flex. In another embodiment, the tubular member is formed from a block polymer of styrene-ethylene-butylene-styrene (SEBS). More details regarding balloon construction and materials are disclosed in assignee's U.S. Pat. No. 5,868,705 and U.S. Pat. No. 6,554,795, each of which is hereby incorporated by reference in its entirety.

The tubular member 204 may be longitudinally stretched to reduce its inner and outer diameters to a particular desired dimension before being mounted on the catheter shaft 202. To minimize the profile of the balloon catheter, the inner diameter may be reduced to substantially the same diameter as the outer diameter of the catheter shaft. After being stretched, the tubular member may be placed in an oven set to 100 degrees Celsius for about 30 minutes. After the tubular member is removed from the oven and has cooled, the tubular member is preferably cut into a length of about 5 to 9 mm, more preferably, about 8 mm.

After the tubular member is placed over the catheter shaft, the bands 206, 208 are placed around the ends of the tubular member 204. The bands are desirably placed over the tubular member such that the ends of the bands are aligned with the ends of the tubular member, as best illustrated in FIG. 3. The bands are preferably substantially rigid in construction and provide a sufficient compressive force to mechanically bond the tubular member to the catheter shaft. As a result, a substantially fluid tight seal is provided between the tubular member and catheter shaft. Although the bands are generally illustrated as thin-walled tubes, it will be understood that the term “band” should include any device having a generally circular or ring shape or being otherwise capable of surrounding a portion of the tubular member.

In one preferred embodiment, the bands 206, 208 are made from a heat shrink material. The bands of heat shrink material are initially sized for placement over the outer diameter of the tubular member. The subsequent application of heat causes the bands to reduce in diameter and thereby form mechanical bonds that firmly secures the tubular member to the catheter shaft and creates a fluid tight seal. In one embodiment, the bands are preferably made of PET that is heated to 300 degrees Fahrenheit for about 10 seconds such that the PET shrinks and tightens around the tubular member to firmly secure the tubular member to the catheter shaft. In one preferred embodiment, the bands have a width of about 0.25 to 2 mm, more preferably about 1 mm, and have a wall thickness of about 0.5 mils. The bands preferably have sufficient compliance to provide the catheter shaft with the desired flexibility during operation.

In addition to using a heat shrinking material, the bands 206, 208 may be applied over the tubular member 204 according to a variety of other techniques. For example, the bands may be tightened by increasing the diameter of the catheter shaft, crimping the bands, compressing the bands or by utilizing a mechanical locking mechanism. Furthermore, the bands may be applied by sliding the bands over the tubular member in a friction fit relationship. Before construction, each of the bands may be provided as separate components.

A variety of other materials may be used in combination with the bands to further enhance the attachment of the tubular member 204 to the catheter shaft 202. For example, the tubular member 204 may be further attached to the catheter shaft by thermal bonding, adhesive bonding, or fusing. To improve the integrity of an adhesive bond, the surface of the catheter shaft 202 may be first treated through plasma etching. One preferred adhesive for use with the device is LOCTITE-4014, which is manufactured by the Loctite Corporation. Any other construction using bands to enhance the bond and form a fluid seal between the tubular member and catheter shaft for creating an inflatable balloon is contemplated to be within the scope of the present invention.

When an adhesive is used in combination with the bands to attach the balloon, the catheter shaft is preferably constructed with a selectively reduced compliance (i.e., greater stiffness) in the regions where the adhesive is applied. The reduced compliance along the catheter reduces the magnitude of the stresses applied to the adhesive material during use.

In various embodiments, the bands may include a radiopaque material for enhanced viewing of the balloon catheter during advancement through the patient's vasculature. In this embodiment, at least a portion of one or more bands may be made of or coated with a radiopaque material, such as, for example, tantalum, gold, platinum, iridium, palladium, rhodium or barium. Alternatively, or in addition, a marker comprising a radiopaque material may be disposed over or under the bands. In another embodiment, a radiopaque material is disposed between the band and the tubular member.

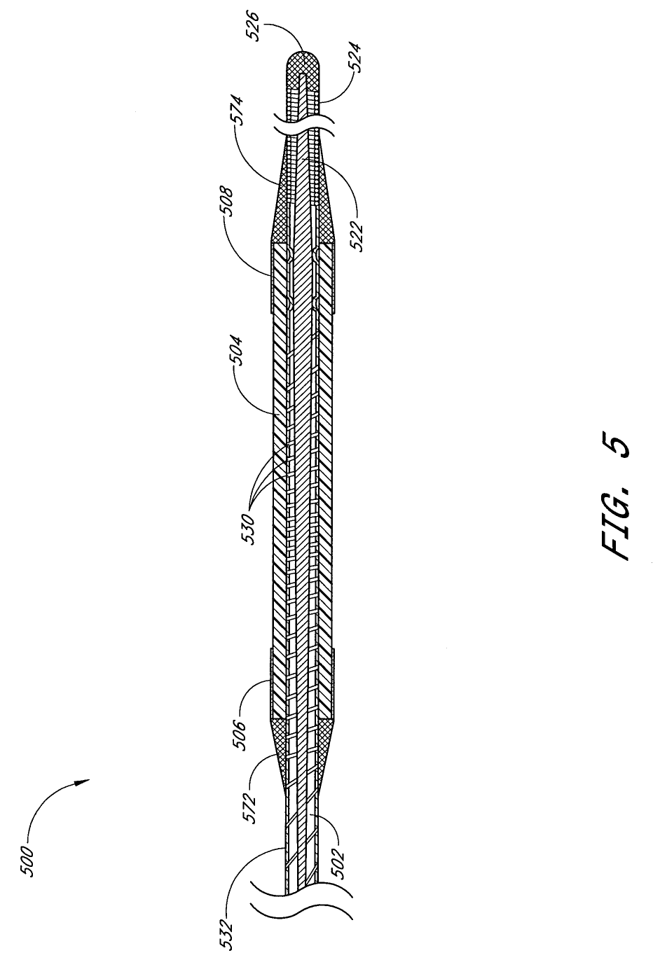

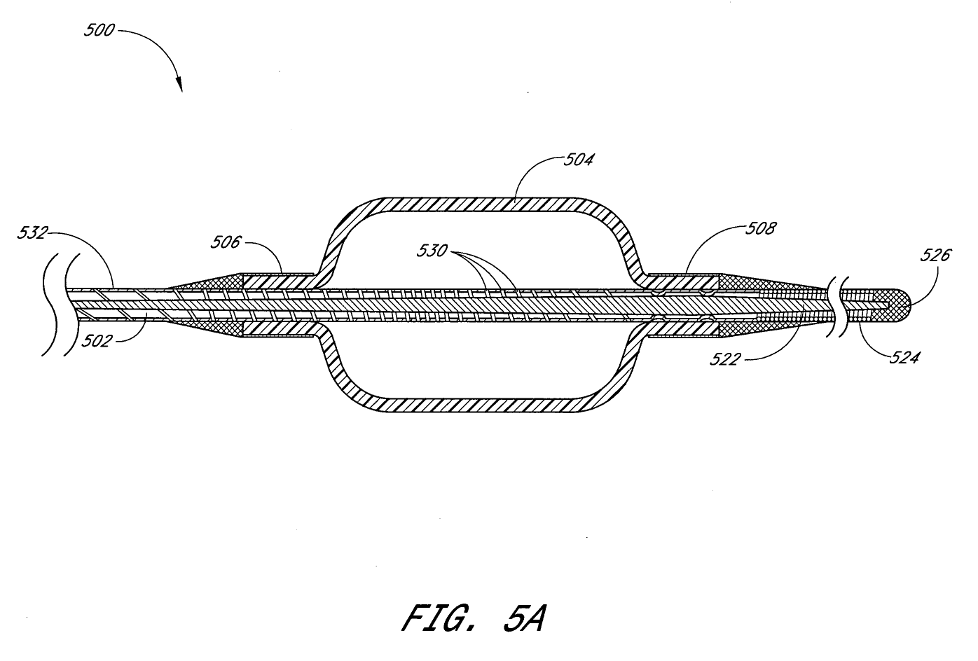

Referring now to FIG. 4B, the bands 206, 208 advantageously provide the tubular member 204 with a compliant section between the bands, and a non-compliant section at the location of the two bands, thereby providing a mechanical seal and preventing fluid loss. The embodiment illustrated in FIG. 4B includes a catheter shaft 202A formed with spiral cuts for enhanced flexibility and for providing an exit port for fluid. The use of spiral cuts in the catheter shaft will be described in more detail below with reference to FIG. 5.

FIG. 5 illustrates a final assembly of another embodiment of a balloon catheter, more preferably an occlusion balloon guidewire such as described with respect to FIGS. 1-2B, wherein a core wire 522 is provided inside the lumen 510 and is crimped to the catheter body 502. A coil 524 extends from the distal end of the catheter body 502, surrounds the core wire 522, and terminates in a rounded cap 526. In one embodiment, the core wire may have one or more tapers, and can extend proximally into catheter body 502. Other details regarding the core wire are discussed in assignee's U.S. Pat. No. 6,355,016, the entirety of which is hereby incorporated by reference.

In the embodiment shown in FIG. 5, the catheter body 502 is formed with spiral cuts 530 to create a coiled configuration, as described in U.S. Pat. No. 6,500,147 and assignee's application entitled FLEXIBLE CATHETER WITH BALLOON SEAL BANDS, application Ser. No. 09/653,217, filed Aug. 31, 2000, now abandoned, incorporated by reference herein. A sleeve or shrink tube 532 is preferably provided over part of the catheter body 502 to cover the spiral cuts 530 proximal to the balloon 504, and extends into the interior of the balloon 504. The balloon 504 is preferably made of a compliant material such as C-Flex, and balloon inflation is provided through the cuts 530 in the catheter body 502, distal to the sleeve 532 within the balloon 504. FIG. 5A illustrates the balloon catheter of FIG. 5 in an inflated condition.

The balloon 504 is applied directly to the catheter body without the use of adhesive, and thus along its proximal end is applied directly against the sleeve 532 and along its distal end is applied directly against the catheter body 502. Adhesive tapers or tapered fillets 572 and 574 are provided adjacent the ends of the balloon 504 to keep the balloon in place and to provide a transition region between the catheter body 502 and balloon 504 at the proximal end and between the balloon 504 and the core wire 522 at the distal end. Bands 506 and 508 secure the balloon 504 to the catheter shaft 502, without adhesive, as generally described above.

Use of the balloon catheter as an occlusion device will now be generally described with reference to FIGS. 5 and 5A. The distal end portion of the balloon catheter 500 is advanced through a patient's vasculature until the tubular member (i.e. balloon) 504 is located distal to a treatment site. An inflation device (not shown) is connected along the proximal end of the catheter shaft 502. When it is desired to inflate the tubular member 504, fluid is injected through the inflation lumen in the catheter shaft 502 and out through the slits 530 in the catheter shaft. As fluid exits the catheter shaft and enters the interior region of the tubular member 504, the fluid causes the tubular member to increase in diameter to a desired size, as illustrated in FIG. 5A. In this application, it is contemplated that the inflatable tubular member may reach a preferred maximum diameter of about 4 mm when expanded. When the therapy is concluded, the tubular member is returned into its collapsed configuration and is withdrawn from the patient's vasculature through the insertion site.

Because the tubular member 504 is attached to the catheter shaft 502 by mechanical bonds (i.e., bands), the volume in the interior region of the tubular member is very predictable and consistent. Accordingly, by controlling the volume of fluid delivered into the interior region of the tubular member, the diameter of the inflated tubular member may be precisely controlled. Furthermore, because mechanical bonds are used to attach the tubular member, the interior volume remains consistent after repeated inflation cycles and the potential for de-lamination is eliminated.

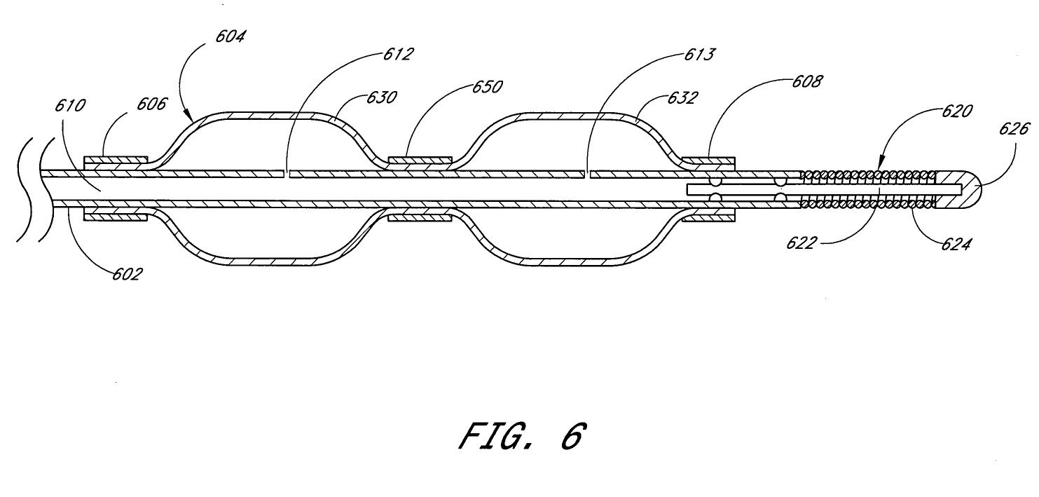

Alternative embodiments of a balloon catheter constructed in accordance with various embodiments of the present invention will now be described with reference to FIGS. 6 through 9B.

FIG. 6 is a side cross-sectional view illustrating a first alternative embodiment of a balloon catheter 600 comprising an proximal band 606, a distal band 608 and further comprising an intermediate band 650 disposed around the tubular member 604 between the proximal and distal bands. The intermediate band 650 may be used to provide the balloon catheter 600 with two separate inflatable balloons 630, 632 along a single catheter shaft 602. Similar to various embodiments described above, the illustrated embodiment of the balloon catheter 600 includes a flexible tip 620 comprising a core wire 622, a coil 624 and a rounded cap 624. It will be appreciated that more than three bands can be used to create inflation zones of desired shapes and sizes.

Multiple balloons on a catheter shaft may be advantageously used for a variety of purposes, such as, for example, improved anchoring in a blood vessel. The balloons may be separately inflatable or may communicate with a common inflation source, as shown in FIG. 6. The balloons may have the same dimensions or may be different. It will be understood by those skilled in the art that, in other embodiments, additional bands may be advantageously disposed along one or more tubular members to provide a wide variety of shapes while remaining within the scope of the present invention.

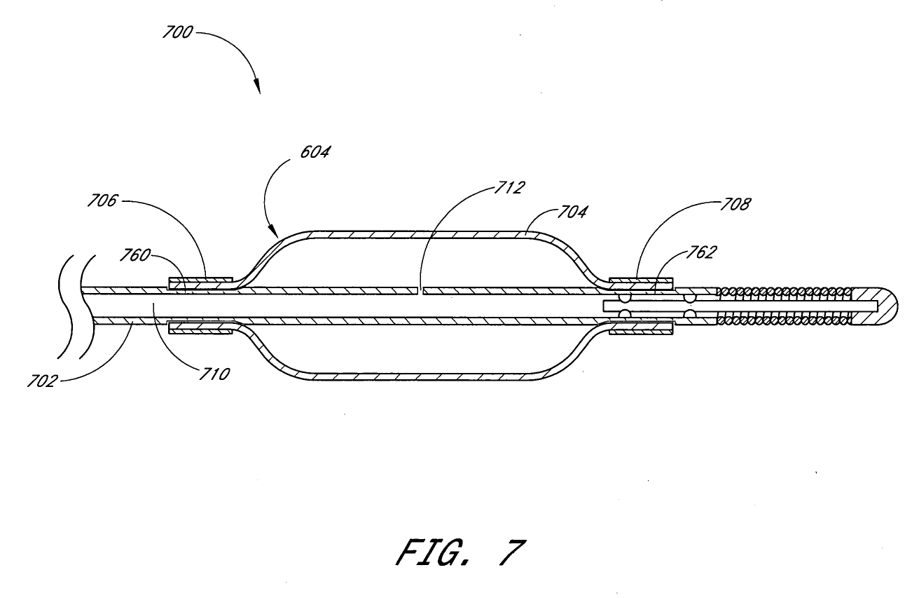

FIG. 7 is a side cross-sectional view of another alternative embodiment of a balloon catheter 700 wherein the catheter shaft 702 is formed with a proximal region of reduced diameter 760 and a distal region of reduced diameter 762. During construction, the proximal and distal ends of the tubular member 704 are placed within the regions of reduced diameter and the proximal and distal bands 706, 708 are applied over the ends of the tubular member. The regions of reduced diameter 706, 708 reduce the profile of the bands for facilitating advancement of the balloon catheter 700 through a patient's vasculature.

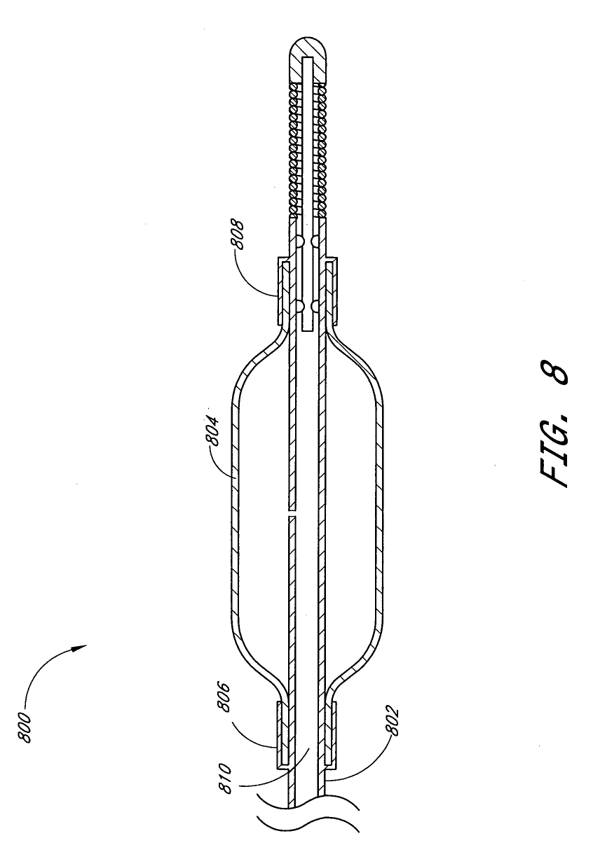

FIG. 8 is a side cross-sectional view of another alternative embodiment of a balloon catheter 800 wherein the proximal and distal bands 806, 808 are integrated as part of the catheter shaft 802. In the illustrated embodiment, the bands 806, 808 are formed as opposing lips extending outward and inward from the outer surface of the catheter shaft 802. The proximal and distal ends of the tubular member are placed between the outer surface of the catheter shaft 802 and the inner surface of the bands. In variations of this embodiment, the bands may comprise a heat shrink material or may be malleable such that they may be crimped inward to hold the ends of the tubular member. Alternatively, a sheath (not shown) may be provided over the bands and catheter shaft to compress the bands inward.

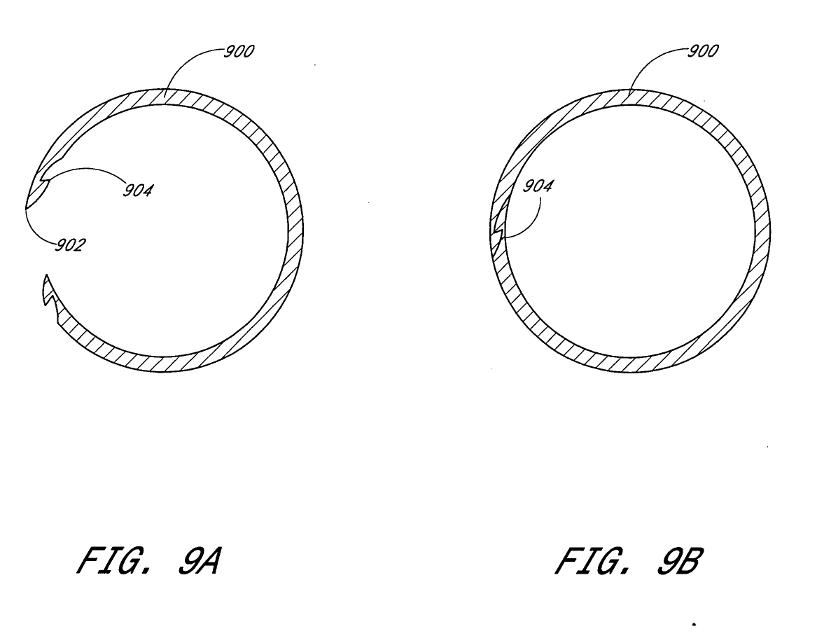

FIGS. 9A and 9B illustrates an alternative embodiment of a band 900 having an open end 902 and being provided with a mechanical locking mechanism 904 or a mechanical tightening means for reducing the inner diameter during construction. FIG. 9A illustrates the band 900 in the unlocked position with a relatively large diameter. In this condition, the band may be slid over a tubular member during construction. FIG. 9B illustrates the band 900 in the locked position wherein the diameter in reduced in size for compressing the tubular member to a catheter shaft.

In view of the above described embodiments, it will be understood by those skilled in the art that the use of bands for attaching a balloon to a catheter shaft has numerous advantages over the use of an adhesive material. For example, the bands provide a balloon catheter wherein the tubular member cannot delaminate from the catheter shaft. This is particularly advantageous for compliant balloon materials, which would previously exhibit poor peel strength when adhesively bonded to different substrates. Furthermore, because the effects of wicking inconsistencies are reduced or eliminated, different balloon catheters constructed in a similar manner will have balloons with substantially identical lengths and diameters upon inflation. As a result, there will be improved consistency and efficacy of the balloons during treatment. In addition, a balloon catheter constructed according to the preferred embodiments will have a balloon that maintains substantially the same length and diameter, even after repeated inflation cycles. Still further, a balloon catheter having increased flexibility may be provided. The increased flexibility is made possible by reducing or eliminating the rigidity of the cured adhesive material.

The above presents a description of the best mode contemplated for an improved balloon catheter according to embodiments of the present invention, and of the manner and process of making and using it, in such full, clear, concise, and exact terms as to enable any person skilled in the art to which it pertains to make and use this device. The embodiments of the balloon catheter described herein are, however, susceptible to modifications and alternate constructions that are fully equivalent. Consequently, it is not the intention to limit this balloon catheter to the particular embodiments disclosed.

What is claimed is:

1. An occlusion balloon guidewire, comprising:
an elongate body having an outer diameter and proximal and distal end portions, said elongate body being formed with a lumen extending along a longitudinal axis;
a compliant balloon having proximal and distal end portions, said compliant balloon being disposed over said distal end portion of said elongate body and being in fluid communication with said lumen; and
first and second bands disposed over said proximal and distal end portions of said balloon, respectively, for adhesiveless coupling of said balloon to said elongate body and for providing a mechanical seal at said proximal and distal end portions of said balloon to prevent fluid loss from an interior region of said balloon.

2. The occlusion balloon guidewire of claim 1, wherein said first and second bands are made of a heat shrink material.

3. The occlusion balloon guidewire of claim 1, wherein said first and second bands are made of PET.

4. The occlusion balloon guidewire of claim 1, wherein said first and second bands have a width of between about 0.25 and 2 mm.

5. The occlusion balloon guidewire of claim 1, wherein said balloon is made of a styrenic block copolymer.

6. The occlusion balloon guidewire of claim 1, further comprising at least one fillet at an end of said balloon for providing a smooth transition region between said tubular member and said elongate body.

7. The occlusion balloon guidewire of claim 1, further comprising a third band located intermediate said first and second bands and being applied over said compliant balloon to form two inflatable regions.

8. A balloon catheter, comprising:
an elongate body having an outer diameter and proximal and distal end portions, said elongate body being formed with a lumen extending longitudinally therethrough;
an expandable tubular member having proximal and distal end portions, said tubular member being disposed over said distal end portion of said elongate body, said tubular member having an inner diameter of about the same dimension as said outer diameter of said elongate body; and
first and second rigid bands disposed over said proximal and distal end portions of said tubular member, respectively, for attaching said tubular member to said elongated body and for providing a mechanical seal at said proximal and distal end portions of said tubular member to prevent fluid loss from the interior of said tubular member.

9. The balloon catheter of claim 8, wherein said first and second rigid bands are made of heat shrink tubing.

10. The balloon catheter of claim 8, further comprising at least one fillet at an end of said tubular member for providing a smooth transition region between said tubular member and said elongate shaft.

11. The balloon catheter of claim 8, wherein said proximal and distal end portions of said tubular member are tapered for providing a smooth transition region between said tubular member and said elongate shaft.

12. The balloon catheter of claim 8, further comprising an adhesive applied between an outer surface of said elongate body and an inner surface of said tubular member along said proximal and distal end portions.

13. The balloon catheter of claim 12, wherein said elongate body has an increased bending stiffness along said proximal and distal end portion for decreasing delamination of said adhesive from said elongated body.

14. A method of manufacturing a balloon catheter, comprising:
providing a catheter shaft having a proximal end portion and a distal end portion and a lumen extending therethrough;
sliding an expandable tubular member over the distal end portion of the catheter shaft, the expandable tubular body having a proximal end and a distal end; and
applying first and second bands over the proximal and distal ends of the expandable tubular member, respectively; to form a mechanical bond that firmly secures the tubular member to the catheter shaft without the use of adhesive between the tubular member and the catheter shaft.

15. The method of claim 14, wherein the first and second bands are made of a heat shrink material.

16. The method of claim 15, wherein forming the mechanical bond comprises heating the bands to cause them to reduce in diameter and tighten around the tubular member.

17. The method of claim 14, wherein the expandable tubular member is made of an elastomeric material.

18. The method of claim 14, further comprising applying a third band at an intermediate location between the proximal and distal ends of the expandable tubular member.

19. The method of claim 14, further comprising applying tapered fillets at the proximal and distal ends of the expandable tubular member.
